(19) United States
(12) Patent Application Publication
(10) Pub. No.: US 2021/0038784 A1
(43) Pub. Date: Feb. 11, 2021

(54) PERCUTANEOUS LONG TERM LEFT VENTRICULAR ASSIST DEVICE AND NON-INVASIVE METHOD FOR IMPLANTING SAME

(71) Applicant: Cardiac Dynamics, LLC, Bloomfield Hills, MI (US)

(72) Inventor: Safwan Kassas, Bloomfield Hills, MI (US)

(73) Assignee: Cardiac Dynamics, LLC, Bloomfield Hills, MI (US)

(21) Appl. No.: 17/079,174

(22) Filed: Oct. 23, 2020

Related U.S. Application Data
(63) Continuation-in-part of application No. PCT/US2019/031777, filed on May 10, 2019.
(60) Provisional application No. 62/934,924, filed on Nov. 13, 2019.
(60) Provisional application No. 62/669,572, filed on May 10, 2018.

(51) Int. Cl. A61M 1/10; A61M 1/12; A61M 27/00

(52) U.S. Cl. CPC A61M 1/1008; A61M 1/122; A61M 2205/0266; A61M 2205/04; A61M 27/002

(57) ABSTRACT

A ventricular assist device for a mammalian heart is disclosed that includes an aortic lumen member, the aortic member communicating with the interior of the aorta, an indwelling pump mechanism and a cardiac lumen member. The cardiac lumen member has at least one lumen tube that is connected to at least one intraventricular clamp that is connected to the inter chamber septum when the ventricular assist device is in the use position and is connected to at least one vertical septal clamp, the ventricular septal clamp is connected to the exterior facing myocardial tissue when the ventricular assist device is in the use position and at least a portion of the cardiac lumen tube extends through one of the cardiac chambers and a portion of the cardiac lumen extends from the ventricular septal clamp to the indwelling pump mechanism.

### CROSS-REFERENCE

This application claims the benefit of U.S. Provisional No. 62/934,924 (Attorney Docket No. 57620-703.102), filed Nov. 10, 2019, the entire content of which is incorporated herein by reference; this application is also a continuation-in-part of PCT/US2019/31777 (Attorney Docket No. 57620-703.601), filed on May 10, 2019, which claims priority to U.S. Provisional Patent Application No. 62/669,572, filed on May 10, 2018, the full disclosures of which are incorporated herein by reference.

### BACKGROUND OF THE INVENTION

The present disclosure pertains to ventricular assist devices. More particularly, the present disclosure pertains to cardiac assist such as ventricular assist devices that can be positioned non-surgically or using minimally invasive techniques.

Heart Failure, often called congestive heart failure, is a condition in which the heart can no longer pump sufficient blood to the rest of the body. Heart failure is a major health problem in the U.S. with hundreds of thousands of cases diagnosed each year. There are a variety of causes for heart failure. The most common cause is coronary artery disease, which is a narrowing of the small blood vessels that supply blood and oxygen to the heart. Other causes of heart failure include congenital heart disease, heart attacks, heart valve diseases and abnormal heart rhythms (arrhythmias).

A variety of surgeries and devices have been developed to treat patients with heart failure, including coronary bypass surgery, angioplasty, heart valve surgery, addition of a pacemaker, or installation of a defibrillator. When treatments no longer work, a patient is said to be in end-stage heart failure. For patients in end-stage heart failure, a heart transplant is often the only possible treatment option. Unfortunately, there is a serious shortage of donors. The annual number of donor hearts remains around 2,000. However, the patients who are qualified to receive and need donor hearts is estimated to be about 16,500. To compensate for this lack of donor hearts, mechanical circulation support systems have been intensively studied and developed. Such mechanical circulation support systems include artificial hearts and ventricular assist devices.

A ventricular assist device (VAD) is a mechanical pump that helps a ventricle to pump blood throughout the body. The VAD pumps the blood from a weakened or diseased ventricle to the aorta or a pulmonary artery. The components of a VAD vary according to the specific device used. In general, a VAD includes a pump, connections to and from the heart, a control system and an energy supply. In some instances, a VAD is used to keep the patient alive until a donor heart is available. Such use is referred to as a “bridge to transplant.” In “destination therapy” a VAD is used in place of a heart transplant to provide a long-term solution for patients that are not eligible for a heart transplant.

Heretofore, placement of ventricular assist devices such as left ventricular devices in the body of the patient requires surgical access to the chest cavity through various methods commonly referred to as open heart surgery with associated co-morbidities. In such procedures, chest access is obtained by opening the chest wall to access the heart. This includes cutting through all or at least a part of the breast bone to open the chest and implant the ventricular assist device. Such procedures are complex and can produce various co-morbidities. Open heart surgeries typically require advanced and complex surgical resources and personnel. Such procedures are so demanding that many individuals who might benefit from ventricular assist therapy are deemed unsuitable for undergoing such aggressive surgical intervention such as those who have conditions that render them has high or prohibitive surgical risk.

Thus, would be desirable to provide a ventricular assist device that could be deployed without invasive surgical procedures. It is also desirable to provide a procedure and device that could assist individuals experiencing heart failure who are not medically eligible for the more aggressive surgical deployment procedures currently available. It is believed that the present disclosure as well as the method set forth in the device set forth in the claims and specification addresses many needs, including but not limited to, a less invasive treatment method for all patients, particularly those who heretofore were considered poor risks for major surgical intervention. It is also believed that the device and method as disclosed herein may support therapies for additional cardiac indications.

### SUMMARY OF THE INVENTION

A ventricular assist device for a mammalian heart is disclosed that includes an aortic lumen member, the aortic member communicating with the interior of the aorta, an indwelling pump mechanism and a cardiac lumen member. The cardiac lumen member has at least one lumen tube that is connected to at least one intraventricular clamp that is connected to the inter chamber septum when the ventricular assist device is in the use position and is connected to at least one vertical septal clamp, the ventricular septal clamp is connected to the exterior facing myocardial tissue when the ventricular assist device is in the use position and at least a portion of the cardiac lumen tube extends through one of the cardiac chambers and a portion of the cardiac lumen extends from the ventricular septal clamp to the indwelling pump mechanism.

In certain embodiments, the aortic lumen member includes an aortic clamp and an aortic lumen tube connected to the aortic clamp. The aortic clamp is connectable with the aorta and communicating with the interior of the aorta when in the ventricular assist device is in the use position. The aortic lumen extends from the aortic clamp and is configured to connect to the indwelling pump device.

In other embodiments, the left ventricle is accessed directly from within the left ventricular cavity in a retrograde manner from femoral artery, through the aortic valve and left ventricular apex. Alternatively, the left ventricle may be accessed from the femoral vein, via a transseptal puncture, through the mitral valve and left ventricular apex, avoiding the need need to go through the right ventricle. In either way, one anchoring port is placed though the left ventricular apex.

A left ventricular apical conduit comprises a frame or scaffold structure, for example a continuous NiTi or other shape memory scaffold, partially or completely covered or encapsulated by an ePTFE membrane. An exposed end of the scaffold (not covered by the ePTFE scaffold) (we plan to have the anchors covered and likely encapsulated by the fabric) is configured to anchors in the passage through left ventricular apex. Even though the continuous scaffold is typically formed as one continuous laser cut piece (Braided formed frame with the same design can also be alternatively considered), the pattern of the cut varies along the length of the scaffold. The portion which passes through the myocardium (referred to as an intramyocardial component) is patterned to provide a radial force sufficient to maintain patency of the passage and should have sufficient crush resistance to accommodate the negative pressure applied by the pump (the external compression caused by the ventricular muscle at the apical level). The “tail” passing from the ventricle to the pump is patterned to accommodate an approximately 90° bend (either pre-shaped with the bend or sufficiently flexible to be bent after placement) while resisting kinking.

An end of the intramyocardial component will have distal barbs or anchors configured to engage an inner side or wall of the myocardium while small flanges or features will be located (at the epicardial (outer) side of the myocardium) a short distance proximally of the distal barbs or anchors and will be configured to prevent migration of the port to into the ventricle. The barbs on the lumen (inner) side of both the ventricle and the aorta can be connected to each other at their tips level to provide a better seal and anchoring.

The left ventricular apical conduit is introduced in a delivery sheath from a subxiphoid location to the apex over a wire intravascularly through the apex then snared from the subxiphoid to create a tract in a low-profile, radially constrained configuration, e.g. being constrained in the delivery sheath, and is released to expand to is enlarged-diameter, bent confirmation to extend from the ventricular apex to the subxiphoid space where the pump is located. The barbs and the flanges are connected to each other to enable better seal and anchoring.

An aortic conduit comprises a frame or scaffold structure covered by or embedded in an ePTFE, dacron, or other membrane in a manner similar to the left ventricular apical conduit. The geometry and compliance characteristics of the aortic conduit will vary, however, to accommodate the path between an outlet end of the pump and the attachment location through a wall of the aorta. The aortic conduit should have sufficient hoop strength to accommodate the out put pressure of the pump. The aortic conduit may be pre-shaped to have an approximately 45° turn to accommodate the path from the pump in the subxiphoid pocket to the attachment location in the aorta. The end of the aortic conduit passing through the aortic wall will have sufficient radial to provide patency. Barbs or other anchors will typically be provided on the luminal side of the aorta and with flanges or other features of the outer side of the aortic wall, enough to prevent migration of the device into the aorta lumen.

In other alternative embodiments, the inner barbs (those located inside the ventricle or inside the aorta) may be formed as a full or partial diamond or other cell to provide improved stability, seal and anchoring. The conduit may comprise or consist of an intraventricular or intra-aortic segment which provides radial force and a tail segment which provides flexibility, anti-kinking and support structure for the fabric. These two segments may be formed continuously as an integrated structure or may be formed separately and connected via bridges. The inner barbs will typically be encapsulated in fabric or other protective material. The outer barbs will typically be exposed and not be encapsulated to enhance deliverability. While exemplary ventricular conduits may have a pre-formed 90° curve and exemplary aortic conduits may have a 45° curve in some instances straight line conduits can be provided sufficient flexibility and anti-kinking to conform most or all curve and trajectory anatomies.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

The various features, advantages and other uses of the present apparatus will become more apparent by referring to the following detailed description and drawing in which:

FIG. 1 is a perspective view of a non-limiting embodiment of a ventricular assist device as disclosed herein in position in a mammalian heart as disclosed herein;

FIG. 2 is a perspective view of a non-limiting embodiment of an aortic lumen member of the ventricular assist device as disclosed herein;

FIG. 3 is a perspective view of a non-limiting embodiment of portion of an embodiment of the cardiac lumen member of the ventricular assist device as disclosed herein;

FIG. 4 is a perspective view of a non-limiting embodiment of an aortic clamp as employed in the ventricular assist device of FIG. 1;

FIG. 5 is a side view of the aortic clamp of FIG. 4 shown in a collapsed delivery configuration;

FIG. 6 is a perspective view of an alternate embodiment of the aortic clamp of FIG. 4 shown in the use configuration;

FIG. 7A is a perspective view of embodiment of an intra-ventricle septal clamp as illustrated in FIGS. 1 and 2;

FIG. 7B is a perspective view of an embodiment of a right ventricle clamp as illustrated in FIG. 1;

FIG. 8 is a side view of the aortic clamp of FIG. 4 shown in a delivery configuration;

FIG. 9 is a side view of an embodiment of the clamp of FIG. 7A as disclosed herein collapsed for delivery;

FIG. 10 is a side view of an embodiment of the clamp of FIG. 7A as disclosed herein in the extended configuration after delivery;

FIG. 11 is an end view of the clamp of FIG. 10;

FIG. 12 is a side view of the clamp of FIG. 10 in position after deployment;

FIG. 13 is a detail view of an embodiment of a balloon expansion mechanism of the intra-ventricle septal clamp of FIG. 7A;

FIG. 14 is a perspective view of the expansion mechanism of the intra-ventricle septal clamp of FIG. 7A;

FIG. 15 is a top perspective view of the expansion mechanism of the intra-ventricle septal clamp FIG. 7A;

FIG. 16A is a graphic depiction of an embodiment of the pericardial inflation step the method of ventricular assist device placement according to an embodiment as disclosed herein;

FIG. 16B is a graphic depiction of an embodiment of the pericardial inflation step of the method of ventricular assist device placement according to an embodiment as disclosed herein;

FIG. 16C is a graphic depiction of an embodiment of the guided intra-ventricular needle insertion step of the method of ventricular assist device placement according to an embodiment as disclosed herein;

FIG. 16D is a graphic depiction of an embodiment of the intra-ventricular conduit insertion step of the method of ventricular assist device placement according to an embodiment as disclosed herein; and

FIG. 16E is a graphic depiction of an embodiment of the indwelling pump placement step of the method of ventricular assist device placement according to an embodiment as disclosed herein;

FIG. 17 is a graphic depiction of an embodiment of a second non-limiting embodiment of a ventricular assist device as disclosed herein in position in a mammalian heart as disclosed herein;

FIG. 17A is a detailed view of the attachment of the left ventricular lumen member to the left ventricle as shown in FIG. 17;

FIG. 18 is a graphic depiction of a left ventricular lumen member of the ventricular assist device of FIG. 17;

FIG. 19 is a graphic depiction of the aortic lumen member of the ventricular assist device of FIG. 17.

FIG. 20 illustrates an alternative aortic lumen member constructed in accordance with the principles of the present invention.

FIG. 21 illustrates an alternative distal scaffold of the ventricular lumen member constructed in accordance with the principles of the present invention.

### DETAILED DESCRIPTION OF THE INVENTION

Disclosed herein is a device and method that can help patients with congestive heart failure increase their cardiac output. It is believed that the device disclosed herein can be employed to help patients with congestive heart failure increase their cardiac output. In many if not all applications, this can be achieved without the need for cardiac surgery. In some embodiments, the device disclosed can be used to provide long term left ventricular assist device percutaneously without subjecting patient to cardiac surgery.

It is believed that the device disclosed herein when in position in a patient can provide improved patient cardiac output, relieve pulmonary congestion, improve blood pressure and thus improve symptoms of heart failure and can be used as a destination therapy or as a bridge towards heart transplantation. It is also believed that the device as disclosed herein can be implanted using less invasive surgical or non-surgical methods which permits the patient to avoid high-risk cardiac surgery.

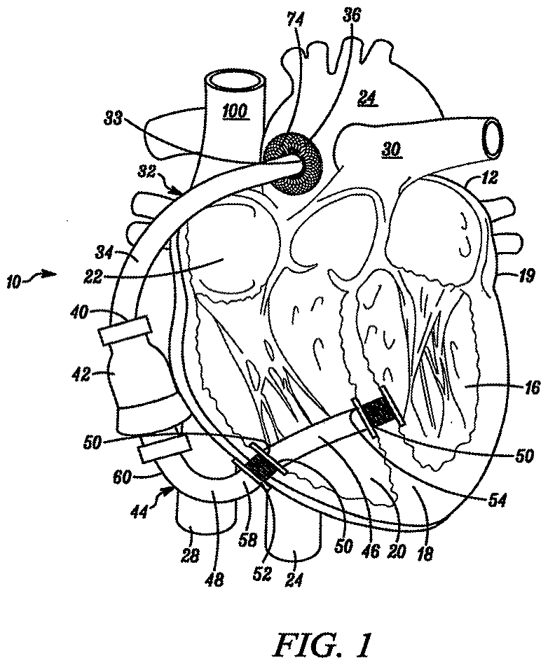

Turning to FIG. 1, a non-limiting embodiment of the ventricular device 10 is depicted in position in a mammalian heart is illustrated. The mammalian heart 12 is illustrated in cross section to illustrate the left atrium 14, left ventricle 16, septum 18, right ventricle 20, and right atrium 22. The aorta 24, in fluid communication with the left ventricle 16 conveys blood away from the heart 12 to remote regions of the associated body. The superior vena cava 26 and inferior vena cava 28 convey blood from remote regions of the body into the right atrium 22 of the heart 12. This returned blood is conveyed into the right ventricle 20 and into the pulmonary artery 30.

The ventricular device 10 as illustrated in FIG. 1, includes an aortic lumen member 32 that defines an aortic fluid channel therein. The aortic lumen member 32 includes an aortic lumen tube 34. The aortic lumen tube 34 that is connected to an aortic clamp 36. In the embodiment depicted, the aortic lumen tube 34 has a first end 38 that is in fluid tight contact with the with the aortic clamp 36 and a second end 40 distal to the first end 38.

The second end 40 of the aortic lumen 34 is connected to a suitable indwelling pump 42 to provide fluid communication through the indwelling pump 42 into the interior of the aortic lumen 34 and into the interior of the associated aorta 24 through aortic clamp 36 such as in the manner described subsequently. In certain embodiments the second end 40 of the aortic lumen 34 can be configured to be releasably connected to the suitable indwelling pump 42 in a fluid tight manner to facilitate implantation of the ventricular device 10 as disclosed herein.

The ventricular assist device 10 also includes a cardiac lumen member 44 that defines a cardiac fluid channel therein. The cardiac lumen member 44 includes at least one cardiac lumen tube and at least one clamp. In the use position, the at least one clamp can be configured to provide fluid access from a chamber of the heart 12. In certain embodiments, the at least one clamp provides fluid access to a ventricle 16, 20. And in certain embodiments, the at least one clamp provides fluid access to the left ventricle 16.

In certain embodiments, the at least one clamp provides access to the left ventricle through the intraventricular septum 18 and at least a portion of the cardiac lumen tube is positioned to transit the right ventricle 20 when the cardiac lumen member 44 is in the use position and at least a portion of the cardiac lumen tube extends outward from the heart 12 connect to an indwelling pump such as indwelling pump 42 in a fluid tight manner. In certain embodiments, the cardiac lumen member 44 of the ventricular access device 10 will include at least two clamps that are located in spaced relationship on the cardiac lumen tube with one clamp located proximate to a first terminal end of the cardiac lumen tube and at least one additional clamp located a spaced distance from the first clamp member and a spaced distance from the second terminal end of the cardiac lumen.

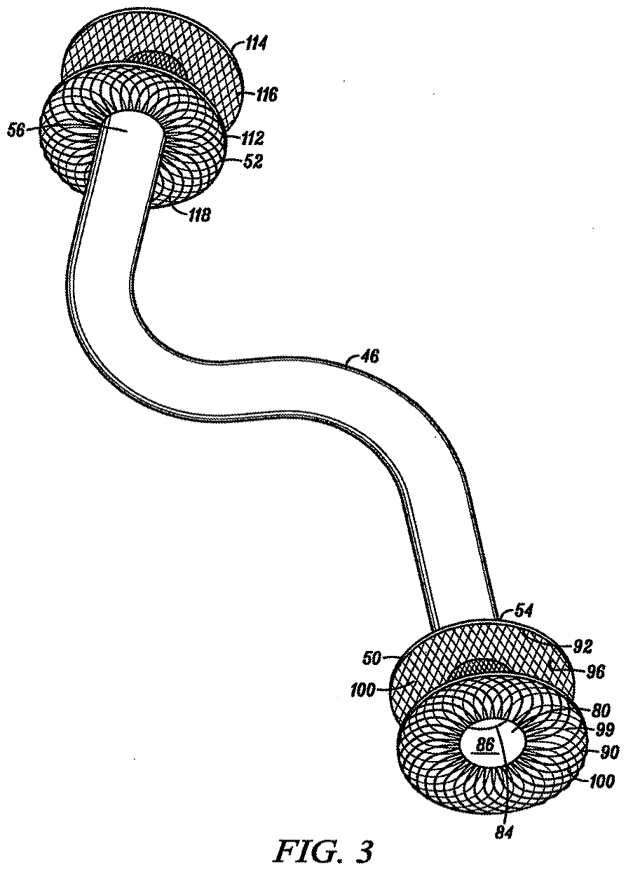

In the embodiment depicted in the various drawing figures, the cardiac lumen member 44 has a cardiac lumen tube 55, this is composed of a first cardiac tube 46 and a second cardiac lumen tube 48 as well at least one intra-ventricle septal clamp 50 and at least one right ventricle septal clamp 52. The first cardiac lumen tube 46 as depicted in FIGS. 1 and 3 has a first end 54 and a second end 56 that is opposed to the first end 54. The first end 54 of the first cardiac lumen tube 46 is connected to the intra-ventral septal clamp 50 and the second end 56 of the first cardiac tube 46 is connected to the right ventricle septal tube 52.

The second cardiac lumen tube 48 has a first end 58 and a second end 60 opposed to the first end 58. In the embodiment depicted in FIG. 1, the first end 58 of the second cardiac lumen tube 48 is connected to the right ventricle septal tube 52 and the second end 60 of the second cardiac lumen tube 48 is connected to the suitable indwelling pump 42 to provide fluid communication to the indwelling pump 42 through the cardiac lumen member 44 from the chamber defined in the left ventricle 16. It is also considered to be within the purview of this disclosure that, in some embodiments, the cardiac lumen 44 be configured from a single tubular member with at least one intra-ventricle septal clamp 50 and at least one right ventricle septal clamp 52 positioned in spaced relationship thereon if desired or required.

One or more of the clamps can be configured with a central tubular member made of Nitinol or other materials, the thickness of the ring is adequate to accommodate the thickness of the ventricular wall. Each side of the central tubular member can be configured with wires or thin plates such as flanges or discs that serve as that serve as anchoring or capturing items when in the deployed or use position. During introduction, the anchoring items such as flanges are oriented in a straight-line position relative to the central tubular member. Where desired or required, the device when in the delivery position can be radially compressed to reduce the delivery profile. When released from the compressed state, the clamp(s) return to their default memory based horizontal line trajectory course or semicircular course. The curving points are at the level of the ring superiorly and inferiorly. When this happens the capturing items will clamp the ventricular wall myocardium between the items from each side of the ring.

The junction between central tubular member and the anchoring items such as flanges can be accomplished by various mechanisms or can be composed of congruous members or fibers.

In certain embodiments such as that depicted herein, it is contemplated that the aortic clamp 36 includes central tubular member 62 that is generally centrally positioned in the aortic clamp 36. The central tubular member 62 has a first end 64 and an opposed second end 66 and a wall member 68 coaxially disposed around a central axis 70 and defines a central shaft that extend therethrough. The aortic clamp 62 also includes at least two flange members that are disposed generally parallel to one another and configured to extend outward from the central tubular member 62 In certain embodiments, one or both plate members can extend from the central tubular member in an orientation that is generally perpendicular to the central axis 70 defined by the central tubular member 62. Other angular orientations relative to the central axis 70 are contemplated and considered to be within the purview of the present disclosure. The central tubular member 62 can have an inner diameter in the use position that is sufficient to convey a sufficient volume of blood to volume of blood from the pump device 42 into the aorta 24 in order to augment regular blood flow into the aorta 24 from the left ventricle 16. Where desired or required, the inner diameter of the central tubular member 62 will be sufficient to allow blood flow rates adequate for supporting or augmenting heart function.

The aortic clamp 36 can include one or more anchor members configured to maintain aortic lumen member 32 of the ventricular assist device 10 in fixed fluid tight contact with the interior channel defined in the aorta 24. In the embodiment depicted in FIGS. 1, 2 and 4, the aortic clamp 36 includes an aortic disc 72 and a pericardial disc 74. The aortic disc 72 can be contiguously connected to one end of the central tubular member 62 and, when in the use position, extends radially outward therefrom at an angle to the central axis 70 that is greater than zero. The pericardial disc 74 can be contiguously connected to one end of the central tubular member 62 opposed to the aortic disc 72 and, when in the use position, extends radially outward therefrom at an angle to the central axis 70 that is greater than zero.

In certain embodiments, the aortic disc 72, when in the use position in the aorta 24, will have an outwardly oriented surface 75 and an opposed inwardly oriented surface 76 relative to central tubular member 62. When the aortic clamp 36 is in position in the aorta 24, the aortic disc 72 is located in the interior channel defined by the aorta 24. Where desired or required, at least a portion of the inwardly oriented surface 76 of the aortic disc 72 is in contact with aortic tissue proximate to the aortic clamp 36 at a location in the channel defined by the aorta 24 while the outwardly oriented surface 75 is oriented f acing the channel defined by aorta 24.

In certain embodiments, the pericardial disc 74, when in the use position in the aorta 24 will have an outwardly oriented surface 78 and an opposed inwardly oriented surf ace relative to central tubular member 62. When the aortic clamp 36 is in position in the aorta 24, the pericardial disc 74 is located on the exterior surface of the aorta 24. Where desired or required, at least a portion of the inwardly oriented surface of the pericardial disc 74 is in contact with the outer surface tissue of the aorta 24 located proximate to the aortic clamp 36 at a location in the channel defined by the aorta 24 through which the central tubular member 62 transits.

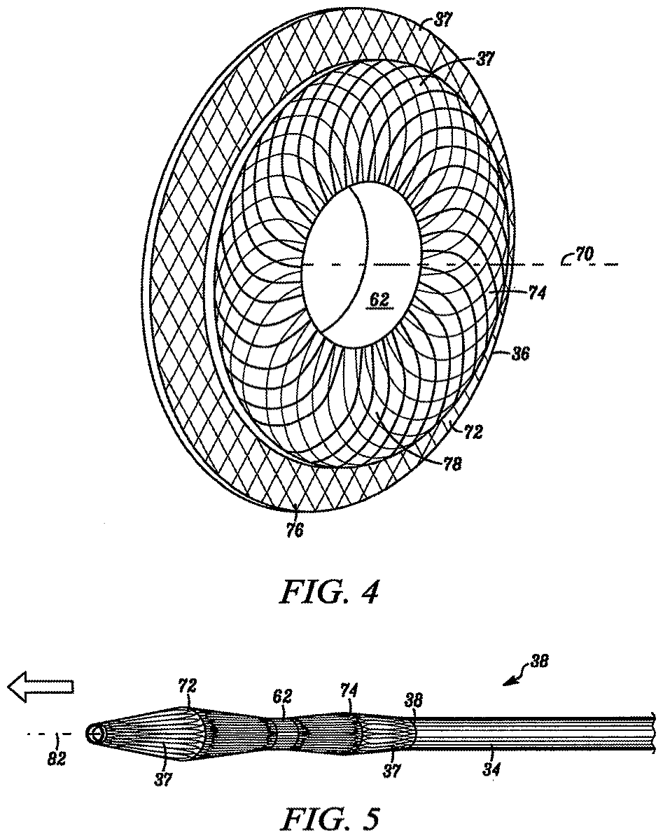

It is contemplated that the aortic disc 72 and the pericardial disc 74 can each have an outer diameter in the use position is sufficient to contact associated tissue in the wall of the aorta 24 in secure relationship therebetween. The respective outer diameters can be equal or can vary as desired or required. In the embodiment depicted, in FIG. 4, the pericardial disc 74 has an outer diameter that is less than the outer diameter of the aortic disc. 72.

While at least one anchor member defined on the aortic clamp 36 can be configured as disc member(s), it is to be understood that other configurations are to be considered with in the purview of this disclosure.

In certain embodiments, it is contemplated that the central tubular member 62 can have mechanical properties similar to an extension spring. Under force, it may increase in length (thereby increasing the distance between the two discs) and when the force is removed, the distance decreases (thereby decreasing the distance between the two discs). In addition, the device can be “shape set” during the manufacturing process to have a desired baseline or resting distance between the two discs.

The aortic clamp 36 can be made, in whole or in part, of collapsible and expanding materials. The ring from each side is connected to a spiral cylinder which when open has a larger diameter than the diameter of the ring. One spiral cylinder is attached to each side of the ring. The spiral cylinder can stretch open into expanded line to reduce deliver profile and when expanded after delivery it will create an accordion like cylinder with central opening to match the ring opening. Accordion cylinders can be covered by fabric.

In the aortic clamp 36 can be composed of any suitable biocompatible material or materials. In certain embodiments, it is contemplated that the material of choice will have one or more of the following properties such as shape memory effect, super elasticity and the like. Where desired or required, the aortic clamp 36 can be composed or whole or in part of a shape memory alloy of nickel and titanium such as nitinol alloys. Non-limiting examples of such materials includes nitinol alloy materials such as Nitinol 55, Nitinol 60 and the like. It is contemplated that, where desired or required, the biocompatible material employed in the aortic clamp 36 can be composed of one or more nitinol alloy materials that are present alone or in combination with other suitable materials. Where desired or required, the aortic clamp 36 can be composed or whole or in part of braided shape memory alloy such as a material such as nitinol.

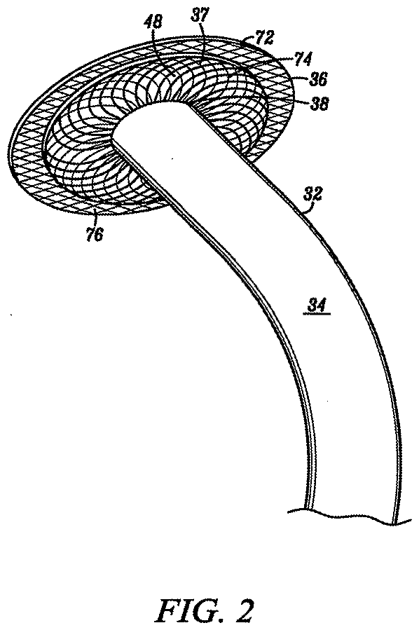

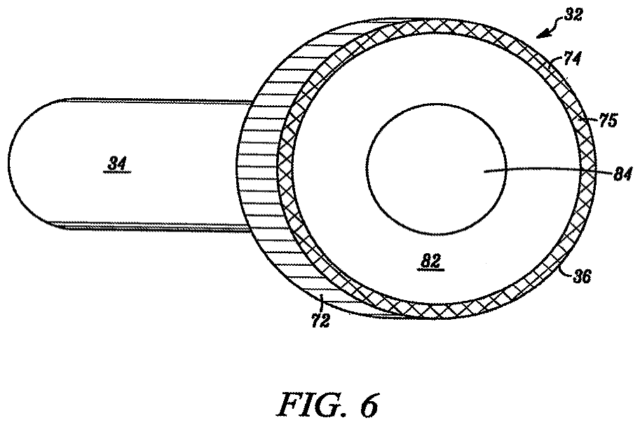

In certain embodiment the biocompatible material or materials such as nitinol can be incorporated in spun or thread material that can be present as braids. In some embodiments, the aortic clamp 36 can have a suitable braided configuration as depicted by thread lines 37 in FIGS. 2, 4 and 5. Where desired or required, the braided structure can be composed thread like material that is made of differing materials in order to modify or tune properties such as shape memory and/or super elasticity and the like. It is also contemplated that material composition and/or thread pattern of the central tubular member 62, aortic disc 72, and pericardial disc 74 can vary from one another in order to address and allow for various characteristics, non-limiting examples of which include radial strength, compressive strength and the like.

Where desired to required, the aortic clamp 36 can be configured with suitable surface materials that can facilitate outcomes such as accurate insertion positioning, maintenance of positions, leakage prevention and the like. Thus, in certain embodiments, the aortic clamp 35 can include suitable biologically compatible mesh or fabric overlaying one or more surfaces of the aortic clamp 36. One non-limiting example of such a configuration is depicted in FIG. 6.

In the embodiment depicted in FIG. 6, a suitable fabric or mesh material 82 can overlay at least a portion of the outwardly oriented surface 75 of aortic disc 74 and can be suitably attached thereto. Where desired or required, the fabric or mesh material 82 can be joined to the material of the aortic lumen 34 either contiguously or by a suitable attachment mechanism such as pre-insertion positioned sutures, biocompatible sealants and the like to provide a continuous interior surface surrounding the channel through which blood can flow into aorta 24.

It is contemplated that the aortic clamp 36 as disclosed herein can be configured to be collapsed to a reduced diameter for purposes of catheter loading and delivery. One method of collapsing is by pulling on a cord that threads through small mini loops on the perimeter of the disc that is facing the delivery access side, so when pulled it will elongate one of the discs and reduce its diameter. Further pulling of the cord will similarly collapse the second disc. The device, once loaded into a catheter, can be delivered into the body to its target location and expanded to its full size.

The aortic lumen tube 34 can be connected to the aortic clamp 36 in a manner suitable to provide a durable fluid tight connection between the two members such that blood can be conveyed from the aortic lumen tube 34 though the central tubular channel 62. In the embodiment depicted in the FIG. 1, the aortic lumen tube 34 employed in the in the aortic lumen member 32 can have an inner diameter that can correspond to the inner diameter of the central tubular channel 62. The first end 38 of the aortic lumen tube 34 be contiguously connected to the outer face 78 of the pericardial disc 74 at a location proximate to the aortic lumen tube 34. The aortic lumen tube 34 can have a length sufficient to permit connection to a suitable pump such as indwelling pump 42. The aortic lumen tube 34 can terminate at second end 40. Where desired or required, the second end of the aortic lumen tube 34 can be configured to facilitate connection to a suitably defined port (not shown) defined in the pump 42 to deliver blood from the pump to the aorta 24.

Where desired or required, it is contemplated that the aortic lumen tube 34 can be composed of a suitable biocompatible material that can be connected to the aortic clamp 36 in a suitable manner. Where desired or required, the biocompatible material employed in the aortic lumen tube 34 can be a suitable woven material and can include material or weave pattern that is kink resistant. In certain embodiments, it is contemplated that aortic lumen tube 34 be integrated with shape memory alloy braids that ex tend from the aortic clamp 36 to form a skeleton structure around or within the body of the aortic lumen tube 34. In certain embodiments, it is contemplated that the aortic lumen tube 34 can be connected to the aortic clamp 36 by sutures or the like. In certain embodiments the connection can be accomplished prior to insertion as by a suitable manufacturer and the aortic lumen member 32 can be deployed into position in the aorta 24 as a preconstructed member.

It is within the purview of this disclosure that an aortic lumen member 32 composed of an aortic tube 34 and aortic clamp 36 can be loaded into a cylindrical delivery device such as a needle, delivery catheter of the like. One non-limiting example of an aortic lumen member configured for delivery to the deployment location is depicted in FIG. 5. In the delivery configuration, the aortic clamp 36 is present in elongated diametrically compressed configuration and is attached to a suitable pericardial wire 82. Insertion can be accomplished by any suitable manner such as by insertion of a suitable surgical catheter through a subxiphoid incision.

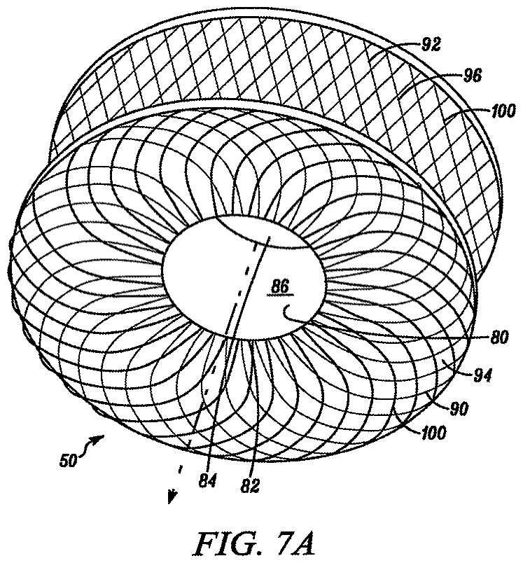

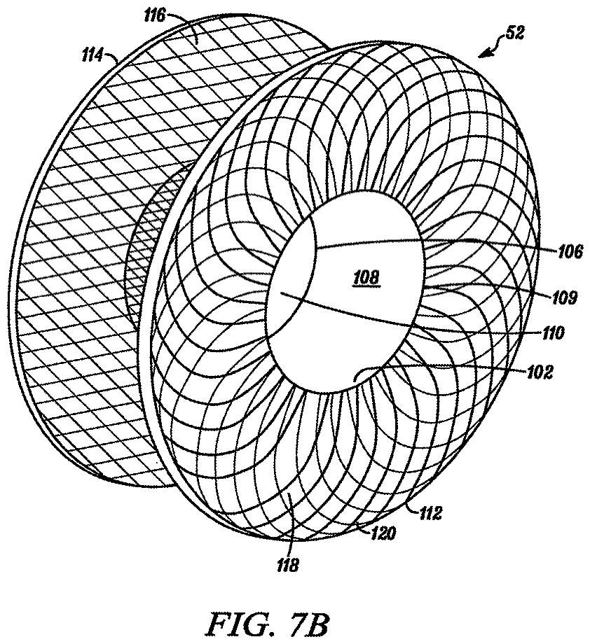

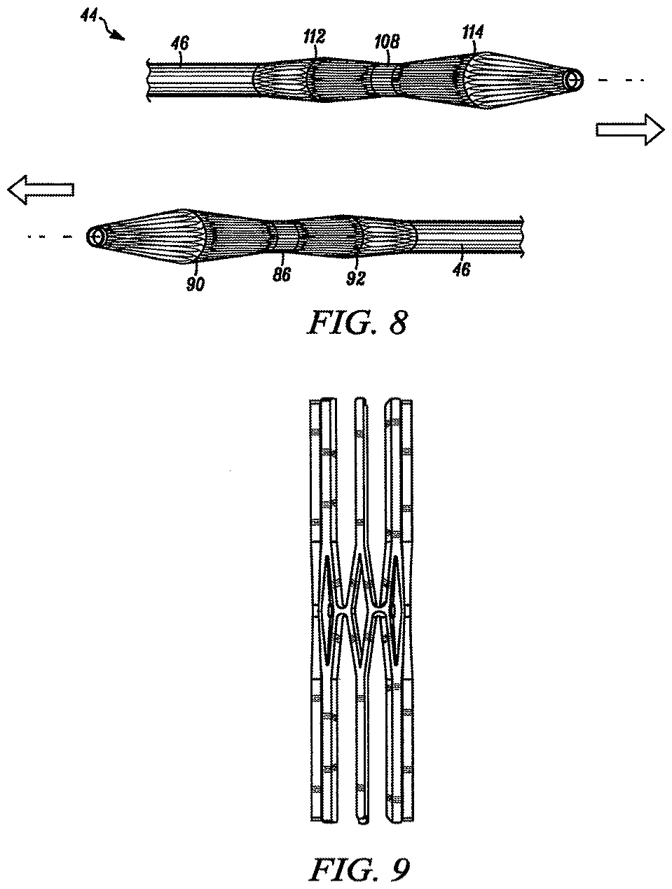

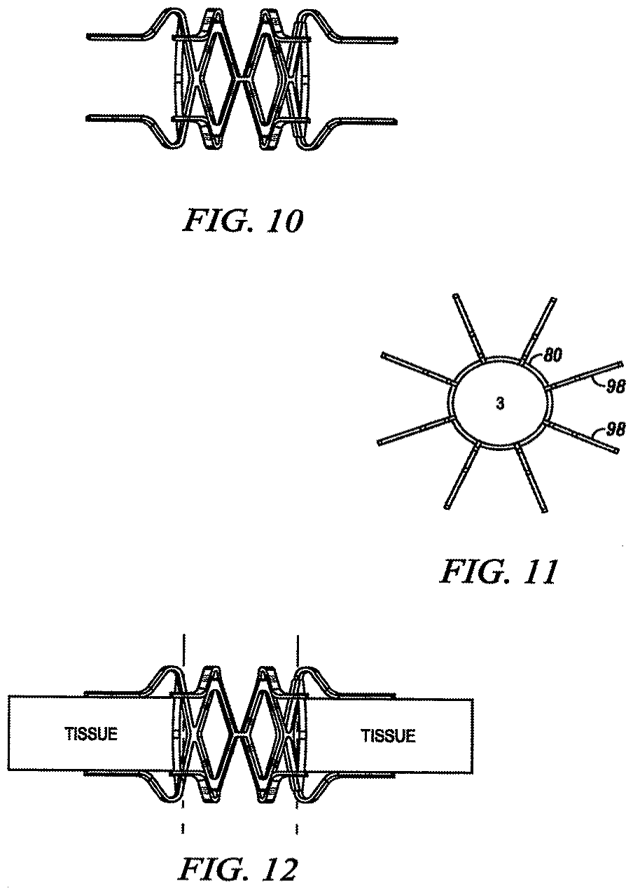

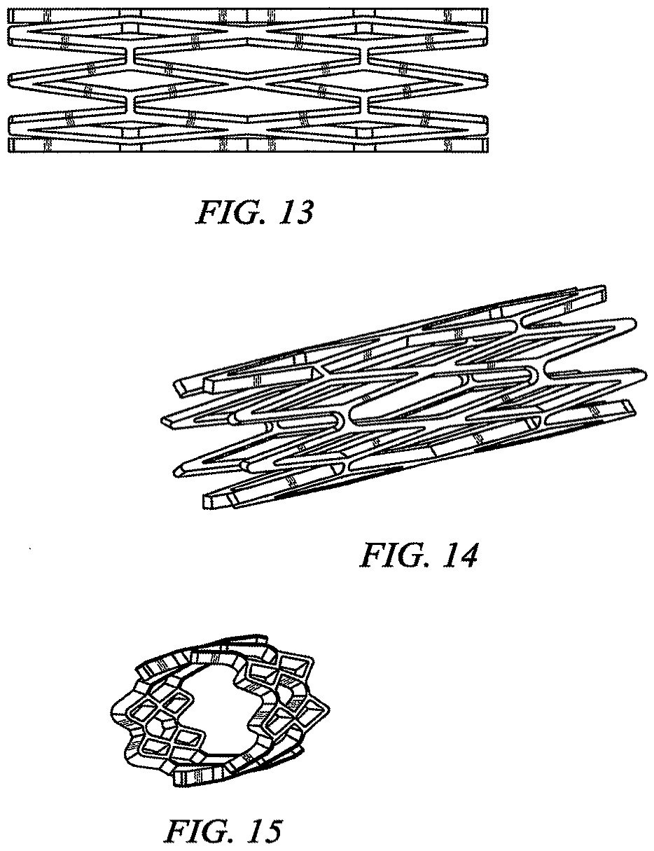

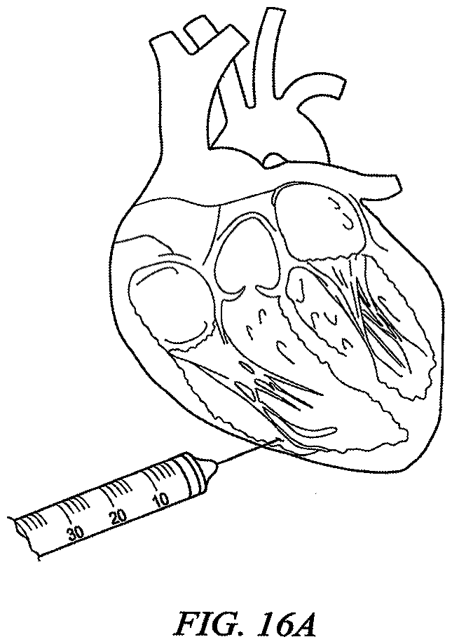

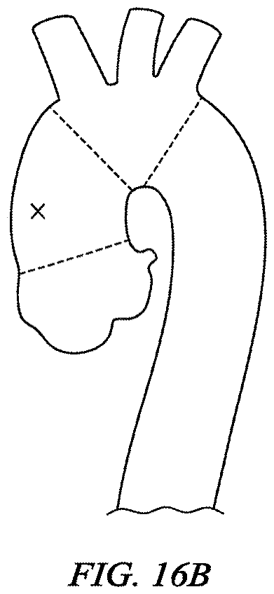

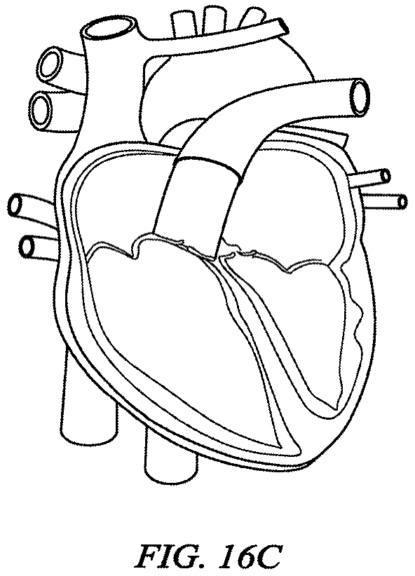

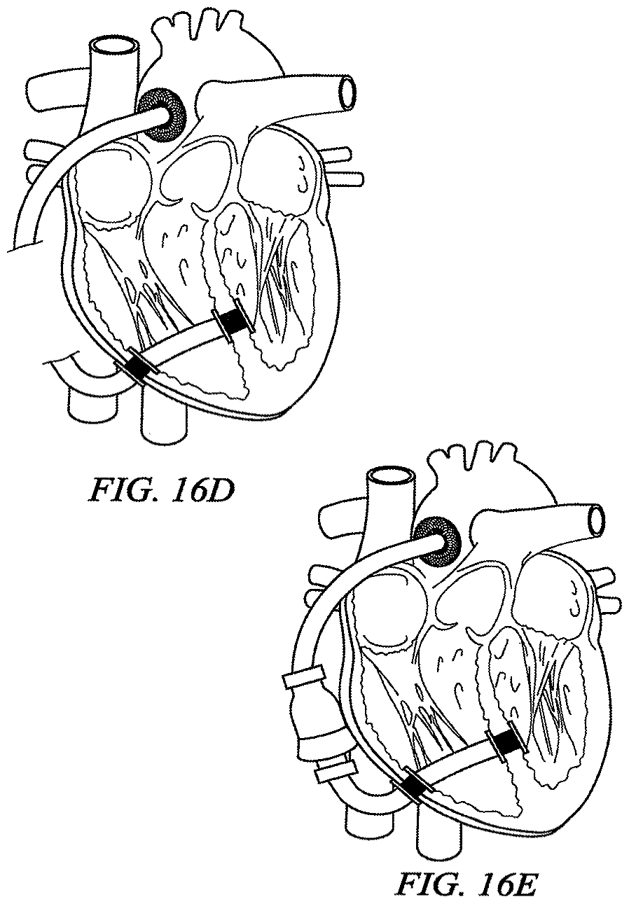

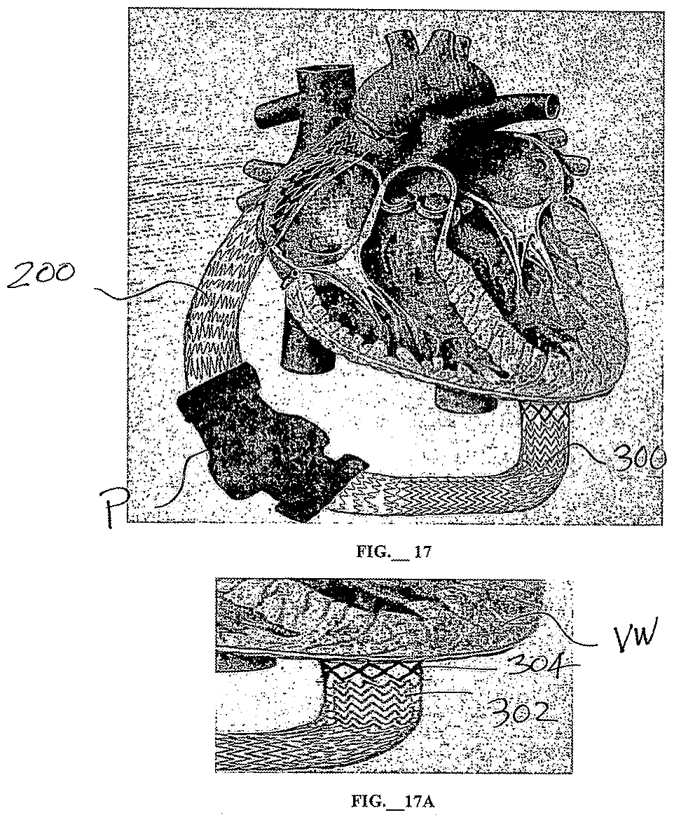

The cardiac lumen member 44 of the ventricular access device 10 can include suitably configured a suitably configured first cardiac tube 46 and a second cardiac lumen tube 48 as well at least one intra-ventricle septal clamp 50 and at least one right ventricle septal clamp 52 that collectively define a cardiac lumen tube 55. An embodiment of the intra-ventricle septal clamp 50 in the deployed configuration is depicted in FIG. 7A. An embodiment of the right vertical septal clamp is disclosed in FIG. 7B.

In certain embodiments such as that depicted herein, it is contemplated that the intra-ventricle septal clamp 50 includes central tubular member 80 at is generally centrally positioned in the intra-ventricle septal clamp 50 and defined a hollow central shaft therein. The central tubular member 80 has a first end 82 and an opposed second end 84 and a wall member 86 coaxially disposed around a central axis 88, the wall member 86 defining the central shaft that extends therethrough. In deployed conditions, the intra-ventricle septal clamp 50 also includes at least two extension members such as the two plate-like disc members depicted in FIG. 7A. The two plate-like extension members can be disposed generally parallel to one another and configured to extend outward from the central tubular member 80.

In certain embodiments, one or both plate members can extend from the central tubular member 80 in an orientation that is generally perpendicular to the central axis 82 defined by the central tubular member 80. Other angular orientations relative to the central axis 82 are contemplated and are considered to be within the purview of the present disclosure. The central tubular member 80 can have an inner diameter in the use position that is sufficient to convey a sufficient volume of blood to volume of blood from the left ventricle 16 ultimately to the indwelling pump device 42 in order to augment regular blood flow into the aorta 24 from the left ventricle 16 through the right ventricle 18. Where desired or required, the inner diameter of the central tubular member 80 will be sufficient to allow blood flow rates adequate for supporting or augmenting heart function.

The intra-ventricle septal clamp 50 can include one or more anchor members configured to maintain cardiac lumen member 44 of the ventricular assist device 10 in fixed fluid tight contact with cardiac septum 18. In the embodiment depicted in FIG. 7A, the intra-ventricular clamp 50 includes a left ventricular flange 90 and a right ventricular flange 92. Wherein desired or required, one or both of the left ventricular flange 90 and the right ventricular flange 92 can be configured as discs as depicted in FIG. 7A. Other configurations can be considered within the purview of this disclosure. The left ventricular flange 90 can be contiguously connected to one end 82 of the central tubular member 80 and, when in the use position, extends radially outward therefrom at an angle to the central axis 88 that is greater than zero. The right ventricular flange 92 can be contiguously connected to an end 84 of the central tubular member 80 opposed to the left ventricular flange 90 and, when in the use position, extends radially outward therefrom at an angle to the central axis 70 that is greater than zero.

In certain embodiments, the left ventricular flange 90 of the intra-ventricular clamp 50, when in the use position in the will have an outwardly oriented surface 94 and an opposed inwardly oriented surface (not shown) relative to central tubular member 80. Similarly, the right ventricular flange 92, when in the use position, will have an outwardly oriented surface (not shown) and an opposed inwardly oriented surface 96 relative to central tubular member 80. When the intra-ventricular clamp 50 is in position in the septum 18, the left ventricular flange 90 is located in the left ventricle 16 and the right ventricular flange 92 is located in the right ventricle 20. Where desired or required, at least a portion of the inwardly oriented surf aces of the respective flanges are contact with septal tissue proximate to the intra-ventricular clamp 50.

It is contemplated that the respective flanges 90 and 92 of the intra-ventricular clamp 50 can each have a size and/or a surface area sufficient to contact associated tissue of the septum 18 placing it in secure relationship therebetween. The respective outer sizes and surface areas can be equal or can vary as desired or required can define a suitable geometric shape. In the embodiment depicted, the respective flanges 90, 92 are shaped as discs and have generally equal sizes. In the embodiment depicted in FIG. 11, one or both of the flanges can be configured in whole or in part as radially extending spokes such as spokes 98. It is also contemplated that one or more of the flanges 90, 92 can be composed in whole or in part of a spiral cylinder or coil (not shown) that when compressed will form a compressed tube but when released will revert to its original pre-determined memory shape so it forms spirals around a central opening. The final spiral from each side form the clamping disc from each side to clamp the associated cardiac tissue in between.

In certain embodiments, it is contemplated that the central tubular member 80 can have mechanical properties similar to an ex tension spring. Under force, it may increase in length (thereby increasing the distance between the two flanges) and when the force is removed, the distance decreases (thereby decreasing the distance between the two flanges). In addition, the device can be “shape set” during the manufacturing process to have a desired baseline or resting distance between the two flanges.

The clamp 50 can be made in whole or in part of collapsible and expanding materials. The ring from each side is connected to a spiral cylinder which when open has a larger diameter than the diameter of the ring. One spiral cylinder is attached to each side of the ling. The spiral cylinder can stretch open into expanded line to reduce deliver profile and when expanded after delivery it will create an accordion like cylinder with central opening to match the ring opening. Accordion cylinders can be covered by fabric.

In certain embodiments, the central tubular member of one or more of the clamps disclosed herein can be made of collapsible and expanding materials. The ring from each side is connected to a spiral cylinder which when open has a larger diameter than the diameter of the ring. One spiral cylinder is attached to each side of the ring. The spiral cylinder can stretch open into expanded line to reduce deliver profile and when expanded after delivery it will create an accordion like cylinder with central opening to match the ling opening. Accordion cylinders can be covered by fabric.

The intra-ventricular septal clamp 50 can also include a suitable flow directing member (not shown) that extends outward from the outwardly oriented surface 94 of the left ventricular flange 90 and is in fluid communication with the central shaft defined by the central tube 80.

In the intra-ventricular septal clamp 50 can be composed of any suitable biocompatible material or materials. In certain embodiments, it is contemplated that the material of choice will have one or more of the following properties such as shape memory effect, super elasticity and the like. Where desired or required, the intra-ventricular septal clamp 50 can be composed or whole or in part of a shape memory alloy of nickel and titanium such as nitinol alloys. Non-limiting examples of such materials includes nitinol alloy materials such as Nitinol 55, Nitinol 60 and the like. It is contemplated that, where desired or required, the biocompatible material employed in the intra-ventricular septal clamp 50 can be composed of one or more nitinol alloy materials that are present alone or in combination with other suitable materials. Where desired or required, the intra-ventricular septal clamp 50 can be composed or whole or in part of braided shape memory alloy such as a material such as nitinol.

In certain embodiment the biocompatible material or materials such as nitinol can be incorporated in spun or thread material that can be present as braids. In some embodiments, the intra-ventricular septal clamp 50 have a suitable braided configuration as depicted by thread lines 37 in FIG. 7A. Where desired or required, the braided structure can be composed thread-like material made of differing materials in order to modify or tune properties such as shape memory and/or super elasticity and the like. It is also contemplated that material composition and/or thread pattern of one or more of the central tubular member 80, left ventricular flange 90, and/or tight ventricular flange 92 can vary from one another in order to address and allow for various characteristics, non-limiting examples of which include radial strength, compressive strength and the like.

In certain embodiments such as that depicted herein, it is contemplated that the right ventricle septal clamp 52 can be configured like the embodiment depicted in FIG. 7B and includes central tubular member 102 at is generally centrally positioned in the right ventricle septal clamp 52 and defining a hollow central shaft therein. The central tubular member 102 has a first end 104 and an opposed second end 106 Located at opposed ends of a central wall member 108 coaxially disposed around a central axis 110, the wall member 108 defining the central shaft that extends therethrough. In deployed conditions, the right ventricle septal clamp 52 also includes at least two extension members such as the two plate-like members depicted in FIG. 7 A. The two plate-like members can be disposed generally parallel to one another and configured to extend outward from the central tubular member 104.

In certain embodiments, one or both plate members can extend from the central tubular member 104 in an orientation that is generally perpendicular to the central axis 110 defined by the central tubular member 104. Other angular orientations relative to the central axis 110 are contemplated and are considered to be within the purview of the present disclosure. The central tubular member 104 can have an inner diameter in the use position that is sufficient to convey a sufficient volume of blood to volume of blood that originates in the Left ventricle 16 and travels through the first cardiac tube 46 that transits the right ventricle 20 into the second cardiac tube 48 and on to the indwelling pump device 42 in order to augment regular blood flow into the aorta 24. Where desired or required, the inner diameter of the central tubular member 104 will be sufficient to allow blood flow rates adequate for supporting or augmenting heart function.

The right ventricle septal clamp 52 can include one or more anchor members configured to maintain cardiac lumen members 44 of the ventricular assist device 10 in fixed fluid tight contact with outer wall of the heart 12 proximate to the right ventricle 20. In the embodiment depicted in FIG. 7B, the right ventricular clamp 52 includes a right ventricular flange 112 and a pericardial flange 114. Wherein desired or required, one or both of the right ventricular flange 112 and the pericardial flange 114 can be configured as discs as depicted in FIG. 7A. Other configurations can be considered within the purview of this disclosure. The right ventricular flange 112 can be contiguously connected to one end 104 of the central tubular member 106 and, when in the use position, extends radially outward therefrom at an angle to the central axis 110 that is greater than zero. The pericardial flange 114 can be contiguously connected to an end 106 of the central tubular member 106 opposed to the pericardial flange 90 and, when in the use position, extends radially outward therefrom at an angle to the central axis 112 that is greater than zero.

In certain embodiments, the pericardial flange 114 of the right ventricular clamp 52, when in the use position in the will have an outwardly oriented surface (not shown) and an opposed inwardly oriented surface 116 relative to central tubular member 102. Similarly, the right ventricular flange 112, when in the use position, will have an outwardly oriented surface 118 and an opposed inwardly oriented surface (not shown) relative to central tubular member 102. When the right ventricular clamp 52 is in position in the septum cardiac wall, the right ventricular flange 112 is located proximate to the outer cardiac wall. Where desired or required, at least a portion of the inwardly oriented surfaces of the respective flanges are contact with cardiac muscle tissue defining the cardiac wall proximate to the right ventricular clamp 52.

It is contemplated that the respective flanges 112 and 114 of the right ventricular clamp 52 can each have a size and/or a surface area sufficient to contact associated tissue of the cardiac wall placing it in secure relationship therebetween. The respective outer sizes and surface areas can be equal or can vary as desired or required can define a suitable geometric shape. In the embodiment depicted, the respective flanges 112, 114 are shaped as discs and have generally equal sizes. In the embodiment depicted in FIG. 11, one or both of the flanges can be configured in whole or in part as radially extending spokes such as spokes 98.

In certain embodiments, it is contemplated that the central tubular member 102 can have mechanical properties similar to an extension spring. Under force, it may increase in length (thereby increasing the distance between the two flanges) and when the force is removed, the distance decreases (thereby decreasing the distance between the two flanges). In addition, the device can be “shape set” during the manufacturing process to have a desired baseline or resting distance between the two flanges.

The right ventricular septal clamp 52 can be made in whole or in part of collapsible and expanding materials. The ring from each side is connected to a spiral cylinder which when open has a larger diameter than the diameter of the ring. One spiral cylinder is attached to each side of the ring. The spiral cylinder can stretch open into expanded line to reduce deliver profile and when expanded after delivery it will create an accordion like cylinder with central opening to match the ring opening. Accordion cylinders can be covered by fabric.

In the right ventricular septal clamp 52 can be composed of any suitable biocompatible material or materials. In certain embodiments, it is contemplated that the material of choice will have one or more of the following properties such as shape memory effect, super elasticity and the like. Where desired or required, the intra-ventricular septal clamp 50 can be composed or whole or in part of a shape memory alloy of nickel and titanium such as nitinol alloys. Non-limiting examples of such materials includes nitinol alloy materials such as Nitinol 55, Nitinol 60 and the like. It is contemplated that, where desired or required, the biocompatible material employed in the right ventricular septal clamp 52 can be composed of one or more nitinol alloy materials that are present alone or in combination with other suitable materials. Where desired or required, the right ventricular septal clamp 52 can be composed or whole or in part of braided shape memory alloy such as a material such as nitinol.

In certain embodiment the biocompatible material or materials such as nitinol can be incorporated in spun or thread material that can be present as braids. In some embodiments, the right ventricular septal clamp 52 have a suitable braided configuration as depicted by thread lines 120 in FIG. 7B. Where desired or required, the braided structure can be composed thread-like material made of differing materials in order to modify or tune properties such as shape memory and/or super elasticity and the like. It is also contemplated that material composition and/or thread pattern of one or more of the central tubular member 102, right ventricular flange 112, and/or pericardial ventricular flange 114 can vary from one another in order to address and allow for various characteristics, non-limiting examples of which include radial strength, compressive strength and the like.

Where desired to required, the intra-ventricular septum clamp 50 and/or the right ventricle septal clamp 52 can be configured with suitable surface materials that can facilitate outcomes such as accurate insertion positioning, maintenance of positions, leakage prevention and the like. Thus, in certain embodiments, the intra-ventricular septum clamp 50 and/or the right ventricle septal clamp 52 can include suitable biologically compatible mesh or fabric overlaying one or more surfaces of the respective clamp. One non-limiting example of such a configuration is depicted in FIG. 6 with regard to aortic clamp 36.

The intra-ventricular septum 50 and the right ventricle septum 52 together with the cardiac tube 46 collectively defines a septal ventricle axis. It is to be understood that the intra-ventricular septum 50 and the right ventricle septum 52 can be similar in design to the aortic clamp 36. In certain embodiments, the central tube of the intra-ventricular septum 50 and/or the right ventricle septum 52 where the central tubular section of each clamp 50, 52 has a length to accommodate the thickness of the ventricular septum and/or the right ventricular wall thickness and the diameter of the central open lumen of one or both can be similar or different from the similar diameter in the aortic clamp 36.

The cardiac tube 46 connects the ventricular face of clamp 52 to the ventricular face of clamp 50. The cardiac tube 46 can be made of the same materials as one or both clamps 50, 52 and covered with a thin impenetrable membrane/fabric or be made entirely of fabric such as PTFE or similar materials.

The septal ventricle axis can be collapsed into a delivery catheter in a similar method used to collapse the aortic clamp 36, one non-limiting example of which is by pulling on a cord that is threaded through mini loops on the outer facing of the right ventricular clamp. The central lumen of the respective members is adequate in size when in the use position is sufficient to allow transfer of blood or fluid from one side of the clamp 50 to the other side of clamp 52 while passing through cardiac tube 46.

The aortic lumen member and cardiac lumen member, when used together, one for withdrawal access to the left ventricle and one for return access to the aorta, along with an appropriate pump, constitute a non-surgical system for left heart flow support.

It is contemplated that he axis tubing connected to a suitable indwelling pump 42. It is contemplated that the tubing can be pre-attached by manufacturer to the outer facing of the right ventricular clamp or can be attached after deployment of item 3 using special connection adaptor.

Where desired or required, one or more of the tubes can include expandable bracelets that are kink resistant. These can be metallic or nonmetallic and when manually stretched can be deployed around aortic tubing and/or the tubes of the cardiac lumen member to provide a kink resistant encasing. Where desired or required, the fabric tubing in the cardiac and/or aortic lumen can be supported by a skeleton of nitinol wire or mesh or of other materials to provide support to the body of fabric and this skeleton connects to the particular clamp discs either as a direct continuation extension or via welding or other methods.

The disclosure contemplates that the length of one or more of the tubing sections can be adjusted based on the size and configuration of the specific heart. It is contemplated that possible mechanisms to provide kink resistance mechanism of fiber tubing might include at least one manual insert from the pump side of the tubing collapsible metallic or plastic plates that are spatially distributed to form a cylindrical tube, or to insert a stent or stent like design to provide a kink resistance tube. These insertable bodies can be introduced into the aortic clamp tubing either from the aortic side or the outflow side. These insertable bodies can also be inserted in the ventricular clamps tubing to provide a kink resistance tube. Fabric tubing can also be supported by integrated Nitinol or other materials wires that are woven within the body of the fiber to provide a supporting structure.

It is contemplated that in certain embodiments, one or more clamps can be made of braided or non-braided nitinol, other metallic materials or non-metallic materials such as but not limited to plastic. One or more clamps can be designed in any shape that can stabilize myocardium or aortic wall through discs or barbs or other mechanism, can be self-expanding or balloon expandable or deployed in their original size. The aortic clamp 36 can access aorta and be delivered surgically or percutaneously in any area or location of the myocardium directly or indirectly. Without being bound to any theory, it is believed that the both the aortic lumen member and the cardiac lumen member can be uniquely configured in a manner that permits them to be introduced percutaneously as through a small incision in the sub-xiphoid region of the patient.

The tubular components of the ventricular assist device 10 as disclosed herein can have a diameter similar to the inner tubular member of the respective clamp(s) and can extend for as long as necessary for the intended purpose. The terminal end of the tubular section can be connected to a device, such as an indwelling pump 42, for withdrawing blood from the heart or returning it to the aorta. The tube may be integrated with the discs (for example, by extending the nitinol wires to form the skeleton of the tube) or may be attached to the indwelling pump 42 as a separate structure.

In to place the left ventricular device as disclosed herein, a sub-xiphoid incision can be made in the patient. Pericardial access can be achieved by any suitable process and a wire placed in the pericardial space assuring that the wire goes superior above RV and not inferior and posterior. Once the wire is in position a sheath can be introduced along the wire into the pericardial space and the pericardium dilated using a suitable dilating fluid such as gas or liquid, non-limiting examples of which are normal saline or carbon dioxide.

Transaortic/pericardium puncture can be achieved using a suitable device such as a cautery on a 0.014 wire using suitable procedures. In one non-limiting example of such a procedure aorta puncture is accomplished from aorta (retrograde through femoral artery) above sino-tubular junction STJ and exit in pericardial space, then wire is snared from pericardial space to exit pericardium in sub-xiphoid area. Alternatively, puncture can be from pericardial space into aorta above STJ and then wire is snared in the aorta and pulled into descending aorta.

As necessary, the cautery wire can be exchanged for as stiff wire at this point in the procedure to allow an ascending aorta/pericardial space rail. A non-limiting example can be a 0.035 stiff wire. The aortic clamp 36 with associated tubing 34 attached constrained in the delivery catheter can be advanced and deployed when the aortic wall is captured between the aortic flange 72 and the pericardial flange 74 to provide a peri-aortic leak free system. Placement of the aortic clamp 36 can be at any suitable location on the aorta. In certain embodiments, placement will be in the ascending thoracic aortic region. After placement the aortic lumen member is temporarily occluded to permit placement of the cardiac lumen member and indwelling pump.

Through a sheath or directly in the pericardial space use a long needle and under echocardiographic and fluoroscopic guidance, using the sub-xiphoid incision as access, the right ventricle and the then the ventricular septum are punctured provide access into the into left ventricle just distal to the papillary muscles. The septal ventricular axis composed of the first cardiac tube 46, intra-ventricular septal clamp 50 and right ventricle clamp 52 is advanced in constrained collapsed state in a delivery catheter over the introduced guide wire. When in position, the deploy intra-ventricular septal clamp 50 is deployed first in a manner such that the septum muscle is securely maintained between the right ventricular flange and the left ventricular flange. Deployment can be accomplished in some embodiments by retraction of the sheath and/or be applying pressure to a suitable wire attached to the intra-ventricular septal clamp 50.

Once the intra-ventricular septal clamp 50 is deployed, the first cardiac tube 46 and the right ventricular clamp can be deployed sequentially. The second cardiac tube 48 is connected to the outwardly facing surface of tight ventricular clamp 52 and is configured to connect to indwelling pump 42. That has been introduced though the sub-xiphoid incision.

Once the inflow tube (second cardiac tube 48) and the outflow tube (aortic tube 34) are connected to the indwelling pump 42, the circuit is complete for ventricular assist function. Once the pump is activated and the temporary occlusion removed blood will flow from left ventricle through intra-ventricular septal clamp 50 through the first cardiac tube 46, right ventricular clamp 52, second cardiac tube 48 axis through the pump 42, aortic tubing 34 aortic clamp 36 and into the ascending aorta above STJ. After blood flow is established and guide wires can be removed, any power pump charging wires can be threaded through the incision and the incision closed.

Referring now to FIGS. 17-19, in alternate embodiments, the entire system with all its components may be encapsulated in a thin layer of ePTFE (expanded polytetrafluoroethylene), dacron, or the like. The implantable cardiac pump P is located in a subxiphoid location, typically in a small pocket created by a surgical cut down. The surgical cut down can be performed by a general surgeon, cardiac surgeon, or trained interventional cardiologist. Percutaneous deployment of the ventricular conduit is typically performed in a cath lab via femoral artery access and subxiphoid intrapericardial access after pericardial insufflation. Deploying the aortic conduit percutaneously in the cath lab without surgery is performed via femoral artery access and intrapericardial access after pericardial insufflation.

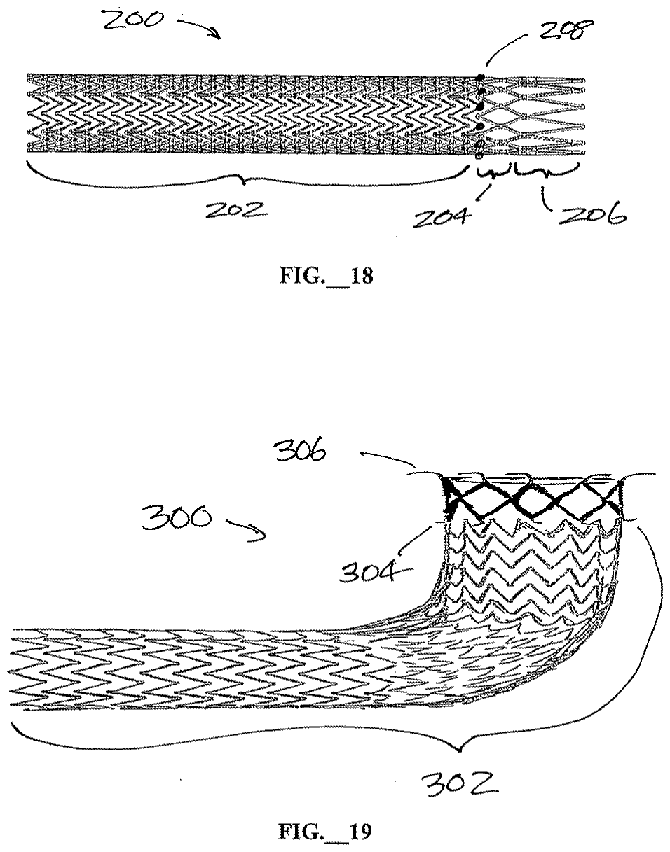

Referring to FIG. 18, an aortic conduit 200 comprises a single laser-cut NiTi (nickel-titanium alloy) tube cut formed as one unit or may be formed as a braided wire or other structure. The aortic conduit 200 is typically patterned to include four regions or zones each of which serves a different function. A tail zone 202 provides a scaffolding structure for the ePTFE cover and forms majority of the length. The scaffold pattern provides flexibility, anti-kinking and assures full expansion of the surrounding ePTFE cover (not shown). Crush resistance is less important for this zone. The aortic conduit is delivered in a radially constrained configuration and is released to be deployed after passing through the penetration in the aortic wall. Upon release from constraint, the tail zone 202 will radially expand to a pre-set memory shape which is about 45-degree to accommodate the path from ascending aorta to the pump P.

Aortic conduit 200 includes a sealing zone 204. The sealing zone 204 will be deployed and centered in a penetration through the ascending aorta wall (FIG. 17). The sealing zone 204 will provide sufficient radially outward force (crush resistance) to overcome compression from the aortic wall penetration.

Aortic conduit 200 further includes anchoring zone 206, typically expanding to form a flange with barbs or the like. When the aortic conduit 200 is collapsed inside the delivery sheath, the barbs are axially aligned with the conduit, When released into the aorta, the barbs expand to anchor the end of the aortic conduit in the aorta. Typically, the barbs are continuation of the scaffold structure and may be interconnected among themselves as well.

Additional small bumps or barbs 208 may be formed on an outer surface of the aortic conduit at the junction between the tail zone 202 and the sealing zone 204 to provide additional anchoring and sealing and to prevent device migration into the aorta.

Ventricular conduit 300 is illustrated in FIG. 19 and is similar to the aortic conduit 200 except that a tail zone bends 302 bends around 90-degree curve to accommodate a path from the apex towards the pump P in the subxiphoid location. A sealing zone 304 expands within a penetration in the left ventricular apex wall, and barbs 306 or other anchors expand against the inner wall of the left ventricle and barbs 308 or other anchors expand against the outer wall of the left ventricle to hold the inlet end of the ventricular conduit 300 in place.

In some embodiments, tethers, pull wire, or the like may be provided, for example through micro holes in the conduit wall, to allow compressibility and collapse of the self-expanding conduits into delivery sheath and allow retrievability of conduits to allow a second attempt deployment if needed.

Where desired or required, the method and device can include the use of suitable adapters such as bracelets, connectors and like that can be applied to one or more of the various tubes to facilitation connection, reduce or eliminate tubing kinking etc.

The invention has been described in connection with what is presently considered to be the most practical and preferred embodiment, it is to be understood that the invention is not to be limited to the disclosed embodiments but, on the contrary, is intended to cover various modifications and equivalent arrangements included within the spirit and scope of the appended claims, which scope is to be accorded the broadest interpretation so as to encompass all such modifications and equivalent structures as is permitted under the law.

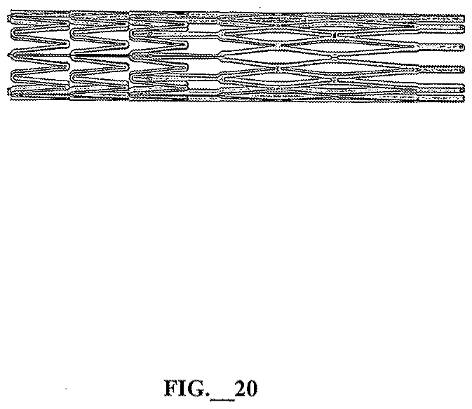

FIG. 20 illustrates an alternative aortic lumen member constructed in accordance with the principles of the present invention.

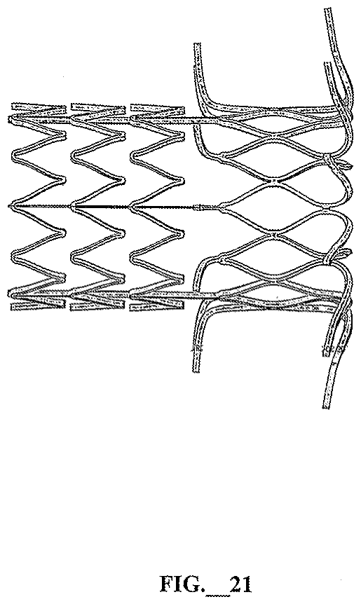

FIG. 21 illustrates an alternative distal scaffold of the ventricular lumen member constructed in accordance with the principles of the present invention.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A system for connecting an indwelling pump mechanism to a left ventricular location and an aortic wall location, said system comprising:
an aortic conduit having one end configured to attach to a penetration in a wall of the aorta and another end configured to attach to an outlet end of the indwelling pump mechanism, wherein the aortic conduit comprises a self-expanding scaffold covered by a membrane; and
a ventricular conduit having one end configured to attach to a penetration in a wall of the left ventricle and another end configured to attach to an inlet end of the indwelling pump mechanism, wherein the ventricular conduit comprises a self-expanding scaffold covered by a membrane.

2. The system of claim 1 wherein the self-expanding scaffolds of the aortic conduit and the ventricular conduit each include a sealing zone, an anchoring zone, and a tail zone.

3. The system of claim 2 wherein the self-expanding scaffolds of the aortic conduit and the ventricular conduit each further include anchor features for securing to an adjacent tissue penetration.

4. The system of claim 3 wherein the tail zone of the aortic conduit has a pre-set bend of from 30° to 60° and the tail zone of the ventricular conduit a pre-set bend of from 75° to 105°

5. A method for implanting an indwelling pump mechanism in a patient, said method comprising:
providing a system as in claim 1;
intravascularly advancing the aortic conduit through a penetration in a wall of the patient's aorta;
expanding the aortic conduit so that the one end anchors in the penetration;
attaching the other end of the aortic conduit to the outlet end of the indwelling pump mechanism;
advancing the ventricular conduit through a penetration in a wall of the patient's left ventricle;
expanding the ventricular conduit so that the one end anchors in the penetration; and
attaching the other end of the ventricular conduit to the inlet end of the indwelling pump mechanism.
